(19) United States
(12) Patent Application Publication
(10) Pub. No.: US 2025/0082231 A1
(43) Pub. Date: Mar. 13, 2025

(54) MEASURING EYE-TISSUE BIOMECHANICS VIA BLINK STIMULATION

(71) Applicant: Alcon Inc., Fribourg (CH)

(72) Inventors: Gangjun LIU, Portland, OR (US); Lingfeng YU, Rancho Santa Margarita, CA (US)

(21) Appl. No.: 18/825,865

(22) Filed: Sep. 5, 2024

Related U.S. Application Data
(60) Provisional application No. 63/582,290, filed on Sep. 13, 2023.

(51) Int. Cl. A61B 5/11; A61B 3/10

(52) U.S. Cl. CPC A61B 5/1128; A61B 3/102; A61B 5/1123

(57) ABSTRACT

Certain aspects of the present disclosure provide systems and methods for measuring eye-tissue biomechanics via blink stimulation. In certain embodiments, a method may be performed by a computer in communication with an optical coherence tomography (OCT) device. The method includes monitoring movement relative to an eye of a patient via one or more images from a camera. The method also includes detecting a blink of the eye of the patient responsive to the monitoring. The method also includes, responsive to the detected blink, recording data resultant from a scan, by the OCT device, of at least a portion of a tissue of the eye. The method also includes measuring a tissue response to the detected blink based on the recorded data from the scan.

### CROSS-REFERENCE TO RELATED APPLICATIONS

This application claims priority to U.S. Provisional Application No. 63/582,290, filed on Sep. 13, 2023, which is hereby incorporated by reference in its entirety.

### INTRODUCTION

Tissue biomechanics, such as corneal biomechanics, can play an important role in understanding, diagnosing, and treating eye diseases such as glaucoma, Keratoconus, and ectasia. Measurement of the biomechanics, however, typically requires external stimulation to a patient's eye.

### SUMMARY

The present disclosure relates to diagnostic systems and methods, and more particularly, to systems and methods for measuring eye-tissue biomechanics via blink stimulation.

In certain embodiments, one general aspect includes a method of measuring eye-tissue biomechanics via blink stimulation. The method may be performed by a computer in communication with an optical coherence tomography (OCT) device. The method includes monitoring movement relative to an eye of a patient via one or more images from a camera. The method also includes detecting a blink of the eye of the patient responsive to the monitoring. The method also includes, responsive to the detected blink, recording data resultant from a scan, by the OCT device, of at least a portion of a tissue of the eye. The method also includes measuring a tissue response to the detected blink based on the recorded data from the scan.

In certain embodiments, another general aspect includes a system for measuring eye-tissue biomechanics via blink stimulation. The system includes an optical coherence tomography (OCT) device. The system also includes a first camera operable to provide one or more images of an eye of a patient. The system also includes a computer communicably coupled to the OCT device and the first camera, where the computer is operable to monitor movement relative to the eye of the patient via the one or more images from the first camera. The computer is also operable to detect a blink of the eye of the patient responsive to the monitoring. The computer is also operable, responsive to the detected blink, to record data resultant from a scan, by the OCT device, of at least a portion of a tissue of the eye. The computer is also operable to measure a tissue response to the detected blink based on the recorded data from the scan.

In certain embodiments, another general aspect includes a computer-program product. The computer-program product includes a non-transitory computer-usable medium having computer-readable program code embodied therein, where the computer-readable program code is adapted to be executed to implement a method. The method includes monitoring movement relative to an eye of a patient via one or more images from a camera. The method also includes detecting a blink of the eye of the patient responsive to the monitoring. The method also includes, responsive to the detected blink, recording data resultant from a scan, by an optical coherence tomography (OCT) device, of at least a portion of a tissue of the eye. The method also includes measuring a tissue response to the detected blink based on the recorded data from the scan.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates an example configuration of an ophthalmic diagnostics system, according to certain embodiments of the present disclosure.

FIG. 1B illustrates another example configuration of an ophthalmic diagnostics system, according to certain embodiments of the present disclosure.

FIG. 2 is a block diagram of various components of the ophthalmic diagnostics system of FIGS. 1A-B, according to certain embodiments of the present disclosure.

FIG. 3 illustrates example aspects of an ophthalmic diagnostics system, according to certain embodiments of the present disclosure.

FIG. 4 illustrates an example of a process for measuring corneal biomechanics via blink stimulation, according to certain embodiments of the present disclosure.

FIG. 5 illustrates an example of a process for measuring corneal biomechanics via blink stimulation in a multi-camera system, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

For the purposes of promoting an understanding of the principles of the disclosure, reference will now be made to the implementations illustrated in the drawings, and specific language will be used to describe the same. It will nevertheless be understood that no limitation of the scope of the disclosure is intended. Any alterations and further modifications to the described systems, devices, instruments, methods, and any further application of the principles of the present disclosure are fully contemplated as would normally occur to one skilled in the art to which the disclosure relates In particular, the features, components, and/or steps described with respect to one implementation may be combined with the features, components, and/or steps described with respect to other implantations of the disclosure. For simplicity, in some instances the same reference numbers are used throughout the drawings to refer to the same or like parts.

Eye-tissue biomechanics, including corneal biomechanics such as corneal stiffness, can play an important role in understanding, diagnosing, and treating diseases such as glaucoma, Keratoconus, and ectasia. Detailed clinical assessment of corneal biomechanics has the potential to revolutionize the ophthalmic industry by providing, for example, personalized LASIK (Laser-Assisted in Situ Keratomileusis) and cataract surgery.

One way to measure corneal biomechanics is by capturing a corneal response to external forces. For example, to measure corneal stiffness, a device may apply a high-pressure air pulse to induce a large corneal displacement (e.g., greater than 2 mm). However, the large corneal displacement typically has a significant nonlinear component that may prevent the device from accurately measuring corneal stiffness. In addition, a deformation amplitude of the cornea is affected by the stability of a source of the air pulse.

The present disclosure describes examples of measuring corneal biomechanics via blink stimulation. An eye-blink process generally involves eyelid movement, specifically a rapid closing and opening of an eyelid. In this fashion, the eyelid movement associated with a blink can be relied upon to produce pressure on a corneal surface of an eye. It is recognized herein that, in various embodiments, the pressure generated by the blink is sufficiently high for use in generating measurements using, for example, optical coherence tomography (OCT) devices. In particular, the pressure produced by the blink typically induces movement or vibration of a cornea of the eye and thus, in various embodiments, can be used as a stimulation method for measuring corneal biomechanics. Advantageously, in certain embodiments, since the pressure generated by the blink is a natural human process, it does not require external stimulation devices. Particular examples will be described in greater detail relative to the Drawings.

For illustrative purposes, the present disclosure describes various examples in relation to measuring corneal biomechanics. However, it should be appreciated that similar principles are applicable to measuring biomechanics for other portions of the eye and/or other tissue.

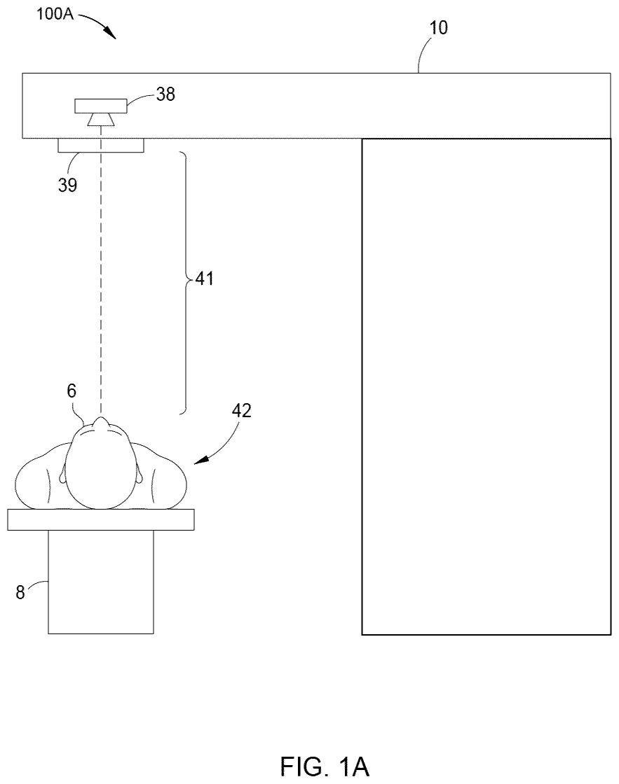

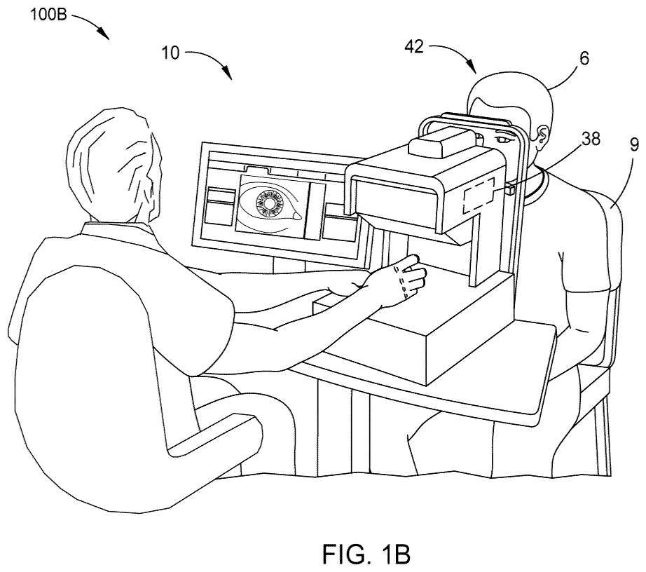

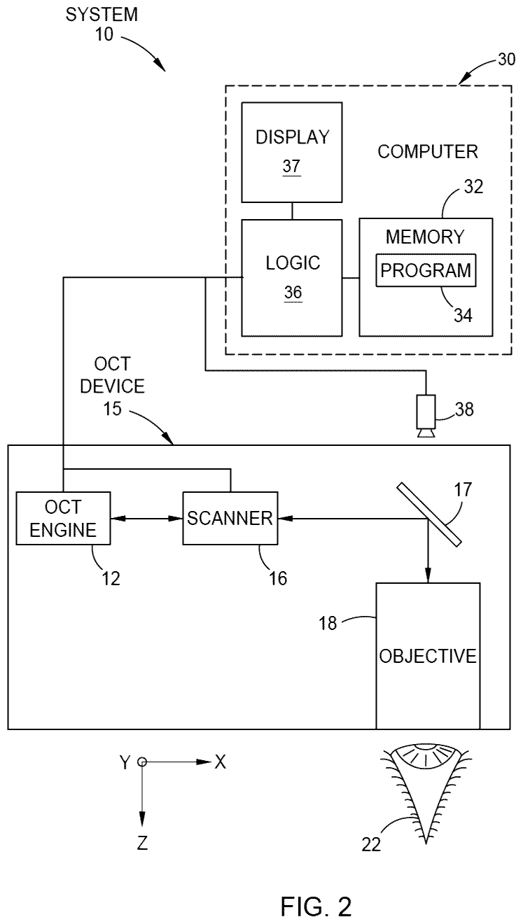

FIGS. 1A, 1B, and 2 illustrate an example of an ophthalmic diagnostics system 10 according to certain embodiments. The ophthalmic diagnostics system 10 may be used for different types of diagnostic and treatment procedures. For example, the ophthalmic diagnostics system 10 may be used for diagnosis or treatment of glaucoma, Keratoconus, and/or ectasia. In addition, or alternatively, the ophthalmic diagnostics system 10 may be used to provide data for personalizing LASIK or cataract surgery.

FIG. 1A shows a configuration 100A of the ophthalmic diagnostics system 10. In particular, FIG. 1A illustrates a head 6 of a patient 42 lying on a bed 8. In the illustrated example, the ophthalmic diagnostics system 10 includes one or more cameras 38 and a part 39 where an imaging light beam exits the ophthalmic diagnostics system 10 and travels through an area 41 towards the patient 42.

FIG. 1B shows a configuration 100B of the ophthalmic diagnostics system 10. In the configuration 100B, the ophthalmic diagnostics system 10 is configured as a desktop imaging system in which the patient 42 sits in a chair 9.

With reference to FIG. 2, the ophthalmic diagnostics system 10 includes an OCT device 15, one or more cameras 38, and a control computer 30, coupled as shown. The OCT device 15 includes controllable components, such as an OCT engine 12, a scanner 16, one or more optical elements 17, and/or a focusing objective 18, coupled as shown. The computer 30 includes logic 36, a memory 32 (which stores a computer program 34), and a display 37, coupled as shown. For ease of explanation, the following xyz-coordinate system is used: The z-direction is defined by the propagation direction of the imaging light beam, and the xy-plane is orthogonal to the propagation direction. Other suitable xyz-coordinate systems may be used.

With particular reference to the OCT device 15, the OCT engine 12 generates and emits an imaging light beam that is guided to tissue of an eye 22 of the patient 42. For example, the imaging light beam may be guided to a corneal surface of the eye 22. The scanner 16 laterally and/or longitudinally directs the imaging light beam. The lateral direction refers to directions orthogonal to the direction of beam propagation, i.e., the x, y directions. The scanner 16 may laterally direct the imaging light beam in any suitable manner. For example, the scanner 16 may include a pair of galvanometrically-actuated scanner mirrors that can be tilted about mutually perpendicular axes. As another example, the scanner 16 may include an electro-optical crystal that can electro-optically steer the imaging light beam.

The longitudinal direction refers to the direction parallel to the imaging light beam propagation, i.e., the z-direction. The scanner 16 may longitudinally direct the imaging light beam in any suitable manner. For example, the scanner 16 may include a longitudinally adjustable lens, a lens of variable refractive power, or a deformable mirror that can control the z-position of the beam focus. The components of the scanner 16 may be arranged in any suitable manner along a beam path, e.g., in the same or different modular units.

One or more optical elements 17 direct the imaging light beam towards the focusing objective 18. An optical element 17 can act on (e.g., transmit, reflect, refract, diffract, collimate, condition, shape, focus, modulate, and/or otherwise act on) the imaging light beam. Examples of optical elements include a lens, prism, mirror, diffractive optical element (DOE), holographic optical element (HOE), and spatial light modulator (SLM). In certain examples, the optical element 17 is a mirror or a dichroic mirror. The focusing objective 18 focuses the imaging light beam towards a portion of the eye 22, such as a corneal surface thereof. In the example, focusing objective 18 is an objective lens, e.g., an f-theta objective.

The OCT engine 12 receives a returned imaging light beam, backscattered from the eye 22, along the opposite direction of the imaging light beam. The OCT engine 12 can be configured to generate an image or images to provide actionable feedback for storage, as described below in detail. For example, in various embodiments, the OCT engine 12 is configured to interferometrically analyze the returned imaging light beam to provide OCT data representing a position-dependent structural property of the eye 22, such as structural property of a cornea thereof. For example, the OCT engine 12 can be configured to provide OCT data representing an image of the cornea at or in the vicinity of the focal position x,y,z and to provide OCT data representing a position-dependent optical density n (x,y,z) of the cornea as well as a position-dependent mass density p (x,y,z) of the cornea.

Although certain examples of the OCT device 15 are described above, it should be appreciated that, in various embodiments, the OCT device 15 can be configured to conduct different types of OCT scans. In an example, in some embodiments, the OCT device 15 can be configured to execute a M-scan. In another example, the OCT device 15 can be configured to execute a B-scan. In yet another example, the OCT device 15 can be configured to execute an MB-scan. In still other examples, the OCT device 15 can be configured to execute a BM-scan. Other examples will be apparent to one skilled in the art after a detailed review of the present disclosure.

The one or more cameras 38 can continuously capture one or more images of the patient 42. For example, the one or more cameras 38 can be focused on the eye 22 for purposes of tracking eyelid movement. In addition, or alternatively, the one or more cameras 38 can be focused on the head 6 of the patient 42 for purposes of tracking head movement. In some embodiments, the one or more cameras 38 can include a first camera for tracking eyelid movement and a second camera for tracking head movement. Examples of the camera 38 include a video, interferometry, thermal imaging, ultrasound, OCT, and head and/or eye-tracking cameras. The one or more cameras 38 deliver image data, which represent recorded images of the eye 22 and/or the head 6, to the computer 30. In some embodiments, the one or more cameras 38 can be an integral part of the OCT device 15, rather than separate as illustrated in FIG. 2.

The computer 30 controls components of the ophthalmic diagnostics system 10 in accordance with the computer program 34. For example, the computer 30 controls components (e.g., the OCT engine 12, the scanner 16, the optical elements 17, and/or the focusing objective 18) to focus the imaging light beam of OCT engine 12 at a desired location on the eye 22, such as a desired location on a corneal surface thereof. The memory 32 stores information used by the computer 30. For example, memory 32 may store images of the eye 22, OCT data, and/or other suitable information, and the computer 30 may access information from the memory 32. In various embodiments, the computer program 34 and its functionality, such as the focusing of the imaging light beam, can be directed by a user such as a medical professional.

In certain embodiments, the computer 30 can measure corneal biomechanics of the eye 22 using blinks as stimuli. In certain embodiments, the computer 30 monitors movement relative to the eye 22 via the image data from the one or more cameras 38. When the computer 30 detects, for example, a closing of the eye 22 followed by an opening of the eye 22, the computer 30 can detect a blink. In response to the detected blink, the computer 30 can record OCT data resultant from an OCT scan of a selected portion of the eye. The recorded OCT dataset can be used by the computer 30 to measure a corneal response to the detected blink.

In certain embodiments, at the beginning of a diagnostic process for the patient 42, the computer 30 can initiate the OCT device 15 and cause the OCT device 15 to continuously scan, for example, the corneal surface of the eye 22. For example, the October 15 device can repetitively scan the same or different portions of the corneal surface. In these embodiments, when the blink is detected, the computer 30 can record data resultant from one or more scans by the OCT device 15 that occurred within a predetermined time interval after the detected blink (e.g., 5 milliseconds to 1 second or 10 milliseconds to 1second).

Alternatively, in some embodiments, instead of continuously scanning, for example, the corneal surface of the eye 22, the OCT device 15 executes OCT scans in response to instructions from the OCT device 15. In these embodiments, the computer 30 can cause the OCT device 15 to execute one or more OCT scans of the corneal surface of the eye 22 immediately upon detection of the blink. Thereafter, the computer 30 can record OCT data resultant from the caused one or more OCT scans and use the recorded data to measure a corneal response to the detected blink.

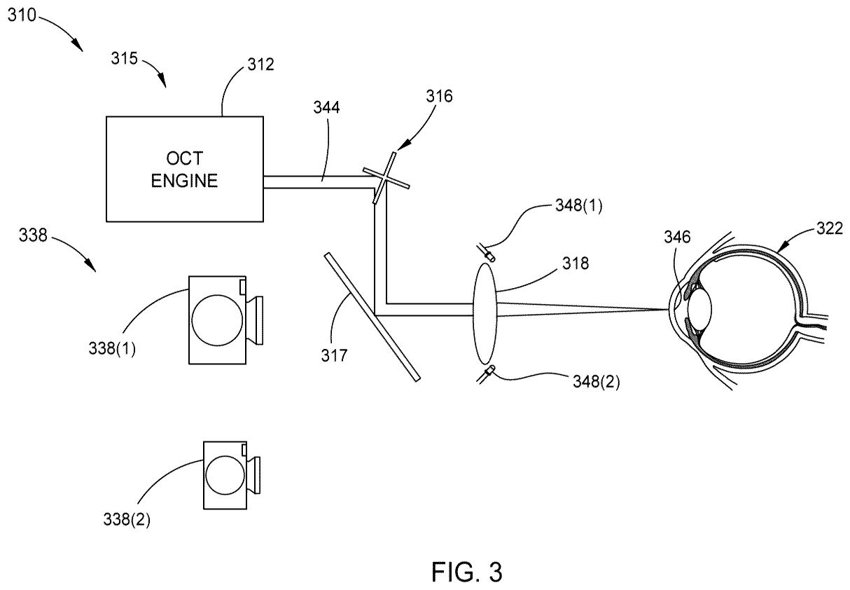

FIG. 3 illustrates an example of an ophthalmic diagnostics system 310. In general, the ophthalmic diagnostics system 310 can include any of the components and functionality described relative to the ophthalmic diagnostics system 10 of FIGS. 1A-B and 2. Similarly, the ophthalmic diagnostics system 10 can include any of the components and functionality described relative to the ophthalmic diagnostics system 310. Therefore, for ease of description, like components of the ophthalmic diagnostics system 10 and the ophthalmic diagnostics system 310 may be periodically referenced interchangeably. For simplicity, the illustration of FIG. 3 focuses on an example OCT device 315 and an example set of cameras 338.

In the illustrated embodiment, the OCT device 315 includes an OCT engine 312, a scanner 316, one or more optical elements 317, a focusing objective 318, and lights 348(1) and 348(2). In similar fashion to the OCT engine 12 of FIG. 2, the OCT engine 312 generates and emits an imaging light beam 344 that is guided to a surface of a cornea 346 of an eye 322, and can thereafter receive a returned imaging light beam, backscattered from the eye 322, along the opposite direction of the imaging light beam 344. In general, the OCT engine 312, the scanner 316, the one or more optical elements 317, and the focusing objective 318 can each operate as described relative to the OCT engine 12, the scanner 16, the one or more optical elements 17, and/or the focusing objective 18, respectively, of FIG. 2.

In the example of FIG. 3, the one or more optical elements 317 are shown as a dichroic mirror, the focusing objective 18 is shown as an objective lens, and the lights 348(1) and 348(2) are shown as light-emitting diodes (LEDs) that direct light toward the eye 322. In various embodiments, the lights 348(1) and 348(2) can improve the quality of image capture by the cameras 338. One skilled in the art will appreciate that the components shown in FIG. 3 can exist in any suitable number or configuration. For example, it should be appreciated that the two lights shown in FIG. 3, namely, lights 348(1) and 348(2), can be modified in quantity, type, and/or configuration to suit a given implementation.

The cameras 338 can operate as described relative to the one or more cameras 38 of FIGS. 1A-B and 2. In the illustrated embodiment, the cameras 338 are shown to include an iris camera 338(1) and a head camera 338(2). The iris camera 338(1) can be focused or fixed on the eye 322, for example, and provide continuous images of the eye 322 to the computer 30. The head camera 338(2) can be focused or fixed on a head of a patient, such as the head 6 of FIGS. 1A-B, and provide continuous images of the head to the computer 30. In some cases, the cameras 338 can be situated behind the focusing objective 318 as shown. In addition, or alternatively, the cameras 338 can be located beside, below or otherwise in proximity to the lights 348(1) and 348(2). For example, the iris camera 338(1) can be located in proximity to the light 348(1) and the head camera 338(2) can be located in proximity to the light 348(2).

In various embodiments, the computer 30 of FIG. 2 is operable monitor eyelid movement relative to the eye 322 via images received from the iris camera 338(1). For example, the computer 30 can detect, as a blink, a closing of an eyelid of the eye 322 followed by an opening of the eyelid. Stated more simply, the computer 30 can detect, as a blink, a closing of the eye 322 followed by an opening of the eye 322.

The computer 30 can determine the closing and opening of the eye 322 in any suitable fashion. In an example, the computer 30 may determine the eye 322 to be closed when an image from the iris camera 338(1) indicates that an iris is no longer visible. Similarly, the computer 30 may determine the eye 322 to be open when an image from the iris camera 338(1) indicates that an iris is visible. Other examples of determining closing and opening will be apparent to one skilled in the art after a detailed review of the present disclosure.

In various embodiments, the computer 30 of FIG. 2 is operable to monitor head movement (e.g., movement of the head 6 of FIGS. 1A-B) via images received from the head camera 338 (2). For example, the computer 30 can establish and/or store a configuration defining a minimum amount of head movement. In various embodiments, if the computer 30 determines that the minimum amount of head movement has occurred within a specified time interval of a detected blink (e.g., within 1 second or 2 seconds before and/or after the detected blink), the detected blink may be ignored, such that the detected blink is not relied upon as a stimulus for measuring corneal biomechanics. In various embodiments, such head movement indicates that no blink has actually occurred and/or that OCT data from the corresponding time interval will not be useful.

In various embodiments, the minimum amount of head movement can be configured or specified in any suitable fashion. For example, with reference to FIGS. 1A-B, in some cases, the minimum amount can be specified in terms of movement and/or orientation of the head 6, for example, relative to a point of reference, such as any previous image or combination of images of the head 6. In other cases, the minimum amount can be specified as a Boolean parameter that indicates whether the head 6 is in motion, such that if motion is detected, the minimum amount of head movement is deemed to be present. In still other cases, the minimum amount of head movement can be specified as a Boolean parameter that indicates whether the head 6 is in a desired alignment (e.g., based on its position and/or orientation in a field of view), such that if the head 6 is deemed to be out of alignment, the specified minimum amount of head movement is deemed to be present. The minimum amount of head movement can also be configured or specified using a combination of the foregoing. Other examples will be apparent to one skilled in the art after a detailed review of the present disclosure.

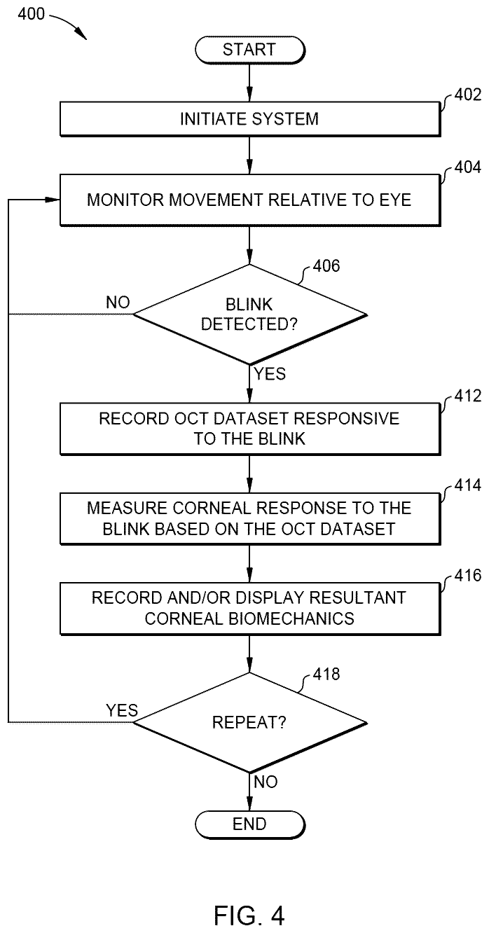

FIG. 4 illustrates an example of a process 400 for measuring corneal biomechanics via blink stimulation. In certain embodiments, the process 400 can be implemented by any system that can process OCT data. Although any number of systems, in whole or in part, can implement the process 400, to simplify discussion, the process 400 will be described in relation to example components of the ophthalmic diagnostics system 10 of FIGS. 1A-B and 2 and the ophthalmic diagnostics system 310 of FIG. 3.

At block 402, the computer 30 initiates the ophthalmic diagnostics system 10. In certain embodiments, the ophthalmic diagnostics system 10 is initiated at the beginning of a diagnostic process for a patient, such as a diagnostic process for the patient 42 of FIGS. 1A-B. The initiating at the block 402 can involve, for example, triggering operation of the OCT device 15, the one or more cameras 38, and/or other components. With respect to the one or more cameras 38, the computer 30 can cause the one or more cameras 38 to begin capturing and providing images.

In some embodiments, with respect to the OCT device 15, the initiation at the block 402 can include the computer 30 causing the OCT device 15 to continuously scan, for example, the corneal surface of the eye 22. For example, the OCT device 15 can be caused to repetitively scan the same or different portions of the corneal surface. In other embodiments, the initiation at the 402 can involve causing the OCT device 15 to enter a standby state in which it is ready to receive OCT scan instructions from the computer 30.

At block 404, the computer 30 monitors patient movement relative to the eye 22 via images continuously received form the one or more cameras 38. For example, as described relative to FIG. 3, the computer 30 can monitor eyelid movement of the patient 42 via the images from the iris camera 338(1).

At decision block 406, the computer 30 determines whether a blink has been detected based on the images from the one or more cameras 38. As described above relative to FIG. 3, a blink may be detected if, for example, the computer 30 detects a closing of an eyelid of the patient 42 followed by an opening of the eyelid. If it is determined at the decision block 406 that no blink has been detected, the process 400 returns to the block 404 and executes as described previously. Otherwise, if it is determined at the decision block 406 that a blink has been detected, the process 400 proceeds to block 412.

At block 412, responsive to the detected blink, the computer 30 records an OCT dataset resultant from one or more OCT scans by the OCT device 15 of at least a selected portion of the eye 22, such as a portion of the cornea 346 of FIG. 3. The recorded OCT dataset can include, for example, OCT data resultant from one or more OCT scans occurring within a predetermined time interval after the detected blink (e.g., 5 milliseconds to 1 second or 10 milliseconds to 1 second). Recording can involve, for example, storing, marking, keeping, and/or otherwise causing the OCT data to persist in association with the patient (e.g., in a patient record or other patient data storage), for example, in the memory 32 or other storage.

Still with reference to the block 412, in some embodiments, as mentioned with respect to the block 402, the OCT device 15 can continuously execute OCT scans of the selected portion of the eye 22. In some of these embodiments, the block 412 can involve the computer 30 selecting OCT data resultant from one or more scans by the OCT device 15 occurring within the predetermined time interval after the detected blink (e.g., 10 milliseconds to 1 second after the detected blink), and storing the selected OCT data in association with the patient as described previously. In addition, or alternatively, the block 412 can involve excluding, from patient data storage, OCT data resultant from OCT scans occurring outside of the predetermined time interval. Exclusion can involve, for example, deleting or discarding such data, making such data available for overwrite, combinations of the foregoing and/or the like.

Still with reference to the block 412, in some embodiments, as mentioned previously, the OCT device 15 can be in a standby state in which it is ready to receive OCT scan instructions from the computer 30. In these embodiments, the recording data at the block 412 can include the computer 30 instructing or causing the OCT device 15 to execute one or more scans of the selected portion of the eye 22, such as a selected portion of the cornea 346 of FIG. 3, and receiving OCT data resultant from the instructed or caused scans. The OCT data that is received can be stored or otherwise kept as described previously.

At block 414, the computer 30 measures a corneal response to the detected blink based on the OCT dataset. In general, the block 414 can include measuring corneal biomechanics of the eye 22 using the detected blink as a stimulus. For example, the computer 30 can generate an OCT amplitude and a phase signal based on the OCT dataset and quantify tissue stiffness (e.g., Young's modulus) based on the OCT amplitude and the phase signal. In some examples, tissue stiffness can be quantified by quantifying one or more vibration properties, for example, of the cornea 346, after the detected blink. The vibration properties can be quantified, for example, via Doppler OCT, amplitude decorrelation, and/or phase decorrelation techniques. The vibration properties can include one or more of vibration amplitude, one or more vibration frequency characteristics, shear-wave propagation features, combinations of the foregoing and/or the like.

At block 416, the computer 30 records and/or displays resultant data from the block 414, such as resultant corneal biomechanics. In various embodiments, the resultant corneal biomechanics can be stored in relation to the patient in the memory 32 or other storage. In addition, or alternatively, the resultant biomechanics can be displayed to a user or operator of the ophthalmic diagnostics system 10.

At decision block 418, the computer 30 determines whether to collect additional corneal biomechanics via additional blink stimulation. If it is determined at the decision block 418 to collect additional corneal biomechanics, the process 400 returns to the block 402 and executes as described previously. Otherwise, the process 400 ends.

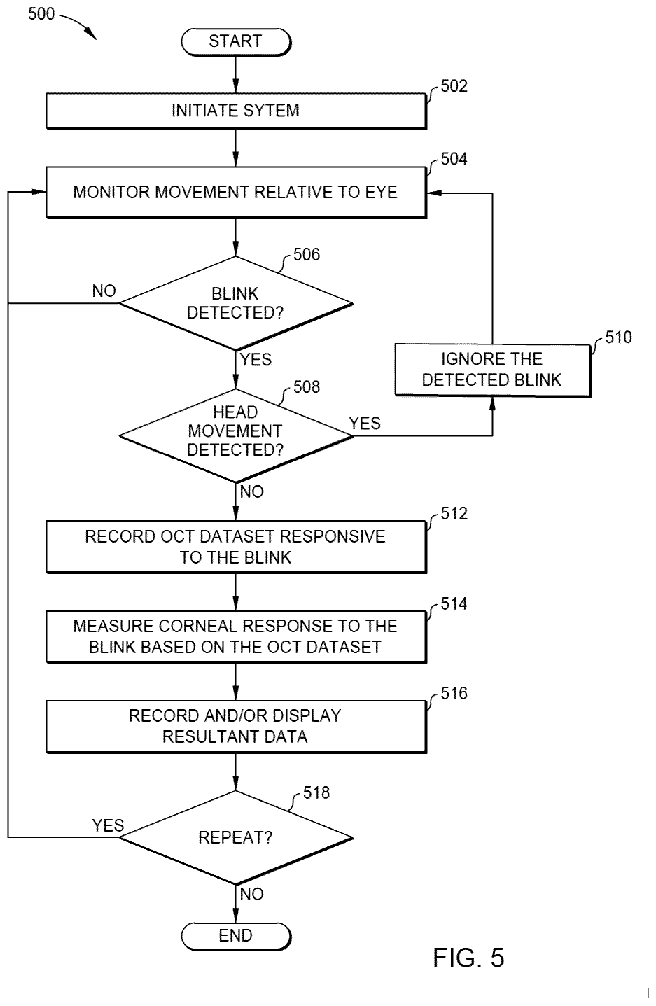

FIG. 5 illustrates an example of a process 500 for measuring corneal biomechanics via blink stimulation in a multi-camera system. In certain embodiments, the process 500 can be implemented by any system that can process OCT data. Although any number of systems, in whole or in part, can implement the process 500, to simplify discussion, the process 500 will be described in relation to example components of the ophthalmic diagnostics system 10 of FIGS. 1A-B and 2 and the ophthalmic diagnostics system 310 of FIG. 3.

At block 502, the computer 30 initiates the ophthalmic diagnostics system 10. In certain embodiments, the ophthalmic diagnostics system 10 is initiated at the beginning of a diagnostic process for a patient, such as a diagnostic process for the patient 42 of FIGS. 1A-B. The initiating at the block 402 can involve, for example, triggering operation of the OCT device 15, the cameras 338, and/or other components. In general, the OCT device 15 can be initiated in the fashion described relative to the block 402 of FIG. 4. With respect to the cameras 338, the computer 30 can cause the iris camera 338(1) and the head camera 338(2) to begin capturing and providing images.

At block 504, the computer 30 monitors patient movement relative to the eye 22 via images continuously received from each of the iris camera 338(1) and the head camera 338(2). For example, as described relative to FIG. 3, the computer 30 can monitor eyelid movement of the patient 42 via the images from the iris camera 338(1). Additionally, also as described relative to FIG. 3, the computer 30 can monitor head movement of the patient 42 via the images from the head camera 338(2).

At decision block 506, the computer 30 determines, based on the images from the iris camera 338(1), whether a blink has been detected. As described above relative to FIG. 3, a blink may be detected if, for example, the computer 30 detects a closing of an eyelid of the patient 42 followed by an opening of the eyelid. If it is determined at the decision block 506 that no blink has been detected, the process 500 returns to the block 504 and executes as described previously. Otherwise, if it is determined at the decision block 506 that a blink has been detected, the process 500 proceeds to decision block 508.

At decision block 508, the computer 30 determines, based on the images from the head camera 338(2), whether head movement of at least a specified minimum amount has been detected in association with the detected blink. In various embodiments, head movement of at least a specified minimum amount can be detected in any suitable fashion inclusive of any of the ways described above relative to FIG. 3. In various embodiments, head movement can be detected in association with the detected blink if, for example, the head movement is detected within a specified time interval of the detected blink, where the specified time interval can span a period of time before and/or after the detected blink.

If it is determined at the decision block 508 that head movement of at least a specified minimum amount has been detected, the computer 30 ignores the detected blink at block 510. In certain embodiments, head movement in association with the detected blink indicates that no blink has actually occurred and/or that OCT data from the corresponding time interval will not be useful. Ignoring the detected blink can involve, for example, excluding, from patient data storage, OCT data resultant from OCT scans occurring within the specified time interval of the detected blink. Exclusion can involve, for example, deleting or discarding such data, making such data available for overwrite, combinations of the foregoing and/or the like. From block 510, the process 500 returns to the block 504 and executes as described previously.

If it is determined at the decision block 508 that no head movement of at least a specified minimum amount has been detected, the process 500 proceeds to block 512. In general, blocks 512-518 execute as described relative to the blocks 412-418 of FIG. 4. The process 500 ends following a negative determination at decision block 518.

In various embodiments, diagnostic systems such as the example diagnostic systems described herein can have various advantages. For example, such a diagnostics system that measures biomechanical properties of diseased or healthy corneas allows for new metrics to be included in treatment planning algorithms to increase predictability and surgeon confidence. Additionally, or alternatively, the ability to produce measurements of corneal biomechanics and apply them to treatment algorithms can support more accurate estimation, prediction, and/or establishment of cataract outcomes from patient-specific surgically induced astigmatism (SIA), Limbal Relaxing Incision (LRI) outcomes from patient-specific calculations, treatment decisions for corneal refractive power, Orthokeratology (Ortho-K) outcomes, treatment and/or diagnosis of dry eye, and/or the like.

The above-disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A method of measuring eye-tissue biomechanics via blink stimulation, the method comprising, by a computer in communication with an optical coherence tomography (OCT) device:
monitoring movement relative to an eye of a patient via one or more images from a camera;
detecting a blink of the eye of the patient responsive to the monitoring;
responsive to the detected blink, recording data resultant from a scan, by the OCT device, of at least a portion of a tissue of the eye; and
measuring a tissue response to the detected blink based on the recorded data from the scan.

2. The method claim 1, further comprising at least one of recording or displaying data resultant from the measured tissue response.

3. The method of claim 1, wherein:
the monitoring movement comprises monitoring eyelid movement of the patient; and
the detecting a blink comprises detecting a closing of the eye followed by an opening of the eye responsive to the monitoring eyelid movement.

4. The method of claim 1, wherein the monitoring movement comprises:
monitoring eyelid movement of the patient via one or more images from a first camera; and
monitoring head movement of the patient via one or more images from a second camera.

5. The method of claim 4, further comprising:
detecting, responsive to the monitoring eyelid movement, a second blink of the eye of the patient;
detecting, responsive to the monitoring head movement, at least a specified minimum amount of movement of the patient's head in association with the detected second blink; and
ignoring the detected second blink responsive to the detected movement of the patient's head.

6. The method of claim 5, wherein the ignoring comprises excluding, from patient data storage, OCT data resultant from one or more OCT scans occurring within a specified time interval of the detected second blink.

7. The method of claim 1, the method further comprising causing the OCT device to continuously scan the at least a portion of the tissue of the eye, wherein the recording data comprises:
selecting OCT data resultant from one or more scans, by the OCT device, occurring within a predetermined time interval after the detected blink; and
storing the selected OCT data in association with the patient.

8. The method of claim 1, the method further comprising causing the OCT device to continuously scan the at least a portion of the tissue of the eye, wherein the recording data comprises excluding, from patient data storage, OCT data resultant from one or more OCT scans, by the OCT device, occurring outside of a predetermined time interval after the detected blink.

9. The method of claim 1, wherein the recording data comprises:
causing the OCT device to scan the at least a portion of the tissue of the eye responsive to the detected blink; and
storing OCT data resultant from the caused scan.

10. The method of claim 1, wherein the measuring a tissue response comprises:
generating an OCT amplitude and a phase signal based on the recorded data; and
quantifying tissue stiffness based on the OCT amplitude and the phase signal.

11. The method of claim 10, wherein the quantifying tissue stiffness comprises quantifying one or more vibration properties of a cornea of the eye after the detected blink.

12. The method of claim 11, wherein the one or more vibration properties comprise at least one of vibration amplitude, one or more vibration frequency characteristics, or shear-wave propagation features.

13. A system for measuring eye-tissue biomechanics via blink stimulation, the system comprising:
an optical coherence tomography (OCT) device;
a first camera operable to provide one or more images of an eye of a patient; and
a computer communicably coupled to the OCT device and the first camera, wherein the computer is operable to:
monitor movement relative to the eye of the patient via the one or more images from the first camera;
detect a blink of the eye of the patient responsive to the monitoring;
responsive to the detected blink, record data resultant from a scan, by the OCT device, of at least a portion of a tissue of the eye; and
measure a tissue response to the detected blink based on the recorded data from the scan.

14. The system of claim 13, further comprising a second camera operable to provide one or more images of a head of the patient, wherein the computer is operable to:
monitor eyelid movement of the patient via the one or more images from the first camera; and
monitor head movement of the patient via the one or more images from the second camera.

15. The system of claim 14, wherein the computer is operable to:
detect, responsive to the monitoring eyelid movement, a second blink of the eye of the patient;
detect, responsive to the monitoring head movement, at least a specified minimum amount of movement of the patient's head in association with the detected second blink; and
ignore the detected second blink responsive to the detected movement of the patient's head.

16. The system of claim 15, wherein the ignoring comprises excluding, from patient data storage, OCT data resultant from one or more OCT scans occurring within a specified time interval of the detected second blink.

17. A computer-program product comprising a non-transitory computer-usable medium having computer-readable program code embodied therein, the computer-readable program code adapted to be executed to implement a method comprising:
monitoring movement relative to an eye of a patient via one or more images from a camera;
detecting a blink of the eye of the patient responsive to the monitoring;
responsive to the detected blink, recording data resultant from a scan, by an optical coherence tomography (OCT) device, of at least a portion of a tissue of the eye; and
measuring a tissue response to the detected blink based on the recorded data from the scan.

18. The computer-program product of claim 17, wherein the monitoring movement comprises:
monitoring eyelid movement of the patient via one or more images from a first camera; and
monitoring head movement of the patient via one or more images from a second camera.

19. The computer-program product of claim 18, the method further comprising:
detecting, responsive to the monitoring eyelid movement, a second blink of the eye of the patient;
detecting, responsive to the monitoring head movement, at least a specified minimum amount of movement of the patient's head in association with the detected second blink; and
ignoring the detected second blink responsive to the detected movement of the patient's head.

20. The computer-program product of claim 19, wherein the ignoring comprises excluding, from patient data storage, OCT data resultant from one or more OCT scans occurring within a specified time interval of the detected second blink.
